(19) United States
(12) Patent Application Publication
(10) Pub. No.: US 2026/0171217 A1
(43) Pub. Date: Jun. 18, 2026

(54) SYSTEM PROVIDING MENTAL THERAPY SERVICES TO ALLEVIATE DEPRESSION AND PSYCHOLOGICAL DISORDERS

(71) Applicant: Korea Institute of Psycho-education, Seoul (KR)

(72) Inventors: Beomyoung KIM, Seoul (KR); Jinhee LEE, Seoul (KR); Yoonsu SONG, Seoul (KR); Yumi HWANG, Seoul (KR); Kyungmi KIM, Seoul (KR); Gangtak CHOI, Ansan-si Gyeonggi-do (KR); Minji KIM, Seongnam-si Gyeonggi-do (KR)

(73) Assignee: Korea Institute of Psycho-education, Seoul (KR)

(21) Appl. No.: 19/423,076

(22) Filed: Dec. 17, 2025

(30) Foreign Application Priority Data
Dec. 17, 2024 (KR) 10-2024-0188620

(51) Int. Cl. G16H 20/70

(52) U.S. Cl. CPC G16H 20/70

(57) ABSTRACT

Embodiments provide a system for providing mental therapy services to alleviate depression and psychological disorders. The system includes a user terminal, wherein the user terminal includes: a user input unit configured to receive user input; and a display unit configured to display a screen of a pre-installed application in the user terminal to provide the mental therapy services to alleviate depression and psychological disorders.

### CROSS-REFERENCE TO RELATED APPLICATION

This application claims priority to Korean Patent Application No. 10-2024-0188620 filed on Dec. 17, 2024, the entire contents of which are herein incorporated by reference.

### TECHNICAL FIELD

Embodiments of the present invention relate to a system for providing mental therapy services to alleviate depression and psychological disorders.

### BACKGROUND

Depression may correspond to a psychological disorder that frequently occurs in modern society and exerts a serious influence on emotional, physical, and social functions of an individual. In general, a depression therapy method may utilize drug therapy, mental therapy, and behavioral therapy, and each method aims for symptom alleviation and recovery. The drug therapy aims to adjust a balance of neurotransmitters in the brain through antidepressants, and the mental therapy aims to organize emotions and thoughts through dialogue with a professional counselor and strengthen problem-solving ability. The behavioral therapy aims to restore physical vitality through improvement of daily habits and enhancement of activities.

However, such therapy methods have respective limitations. The drug therapy depends on neurotransmitter adjustment, thus involving a high possibility of side effects and a risk of tolerance and dependence occurring during a long treatment period. It is frequently difficult to consistently proceed with the mental therapy due to the therapy cost burden and time constraints. In addition, the behavioral therapy essentially requires voluntary participation and the will of a patient, yet a depressed patient may frequently experience difficulty in consistently proceeding with a therapy process due to decreased motivation and lethargy. Due to such problems, existing depression therapy methods exhibit limited therapy effects and exhibit limitations in inducing voluntary recovery motivation of a patient.

### SUMMARY

Embodiments provide a system for providing mental therapy services to alleviate depression and psychological disorders, which overcomes limitations in existing therapy methods and provides a self-healing-based systematic learning environment in which a patient is able to receive recovery motivation on one's own.

Embodiments provide a system for providing mental therapy services to alleviate depression and psychological disorders, which may provide learning data classified into a mental part and a gender part in a customized manner based on gender, thereby supporting a patient to understand principles of emotions and improve an emotional state through learning data suitable for a situation of the patient.

Embodiments provide a system for providing mental therapy services to alleviate depression and psychological disorders, which may include repetitive and systematic processes such as practice tasks, emotion recording, and review writing based on daily learning data, thereby establishing an environment allowing a patient to actively participate in a therapy process, and may repeatedly provide the same curriculum after completion of a the first learning cycle and provide modified review-writing questions to allow the patient to newly recognize learning data, thereby maximizing the learning effect while preventing the patient from feeling boredom.

Embodiments provide a system for providing mental therapy services to alleviate depression and psychological disorders, which may allow a patient to proceed learning and practice tasks by investing a short time each day to experience improvement of an emotional state and psychological stability, and furthermore allow the patient to clearly recognize a recovery state of the patient and determine whether further learning or repeated learning is required on one's own by visually providing a learning effect at a completion time point of a third learning cycle, thereby presenting a new self-healing-based depression therapy method for effectively resolving problems in existing therapy methods.

In one general aspect, provided is a system for providing mental therapy services to alleviate depression and psychological disorders, the system including: a user terminal, wherein the user terminal includes: a user input unit configured to receive user input; and a display unit configured to display a screen of a pre-installed application in the user terminal to provide the mental therapy services to alleviate depression and psychological disorders.

The system may include: a server connected to the user terminal through a wired or wireless network, wherein the server includes: a storage unit configured to store data of the application; a communication unit configured to transmit and receive the data to and from the user terminal; and a control unit configured to control at least one of operations of the user terminal and the server, and wherein the control unit is configured to: control storing of learning data classified into a mental part and a gender part, and provide mental-part learning data and gender-part learning data classified as male-use or female-use based on gender data input by a user according to the same curriculum order; provide the user terminal with the mental-part learning data classified as male-use or female-use based on user gender; provide the user terminal with the gender-part learning data classified as male-use or female-use based on user gender after learning of the mental-part learning data; provide the user with practice tasks performable in daily life, and record the data through a user input unit for receiving information on a practice-task execution state; and control a daily learning process to be completed by displaying an input screen on the user terminal for emotion recording and review writing after completion of learning of the gender-part learning data and the mental-part learning data.

The control unit may be configured to: sequentially provide the mental-part learning data and the gender-part learning data by defining the pre-stored mental-part and gender-part learning data each for 50 days as one cycle on a daily basis; provide each learning data by classifying each of the mental-part and gender-part learning data as male-use content or female-use content based on user gender; repeatedly provide the same learning data for second and third learning cycles when a first learning cycle is completed; classify emotion-record data, learning-response data, and practice-task data input daily by the user for each cycle to store the data in the storage unit; and identify a learning effect based on comparison among the data stored for each cycle when each cycle is completed.

The control unit may be configured to: load question data used for review writing in the first learning cycle from the storage unit, extract major structures (e.g., a subject-verb-object (SVO) structure) included in each question, replace core words among the extracted major structures into substitutable vocabulary from a synonym database, and rearrange a word order based on substituted words or inserting adverbs, adjectives, or additional phrases to enable the same question to be recognized differently by the user, thereby generating a plurality of question data to be used for review writing in the second learning cycle in which sentence structures of the question data used for review writing in the first learning cycle are changed; and execute natural-language similarity evaluation between the plurality of question data and the question data used for review writing in the first learning cycle to identify questions whose similarity is equal to or lower than a predetermined threshold as final transformed question data, and provide the final transformed question data to the user terminal.

The control unit may be configured to: collect the emotion-record data, review data, and practice-task execution-state data input through the user input unit, classify the data based on the learning date and cycle, and store the classified data in the storage unit; and identify response data corresponding to the same question content for each cycle, calculate emotional-state change trends and practice-task execution rates, and output a warning message to the user terminal when at least one of negative emotion states or practice-task non-execution occurs.

The control unit may be configured to: evaluate a learning-progress state of the user based on the data stored after completion of the third learning cycle; calculate emotional-state change trends and identify the practice-task execution rates and response-data consistency by comparing the emotion-record data of the first, second, and third learning cycles to quantitatively derive the learning effect of the user; and output the emotional-state change trends and practice-task execution rates of the user to the user terminal by visualizing the trends and the rates in graph and summary-text forms based on analysis results of the learning effect.

### BRIEF DESCRIPTION OF THE DRAWINGS

FIG. 1 is a diagram illustrating a schematic configuration of a system for providing mental therapy services to alleviate depression and psychological disorders according to an embodiment of the present invention.

FIG. 2 is a diagram illustrating a detailed configuration of the system for providing mental therapy services to alleviate depression and psychological disorders according to an embodiment of the present invention.

FIG. 3 is a diagram illustrating a learning flowchart of the system for providing mental therapy services to alleviate depression and psychological disorders according to an embodiment of the present invention.

### DETAILED DESCRIPTION OF EMBODIMENTS

Hereinafter, an embodiment of the present invention is described in detail with reference to the accompanying drawings. In the drawings, the same reference numerals or signs refer to components that perform substantially the same functions, and sizes of respective components in the drawings may be exaggerated for clarity and convenience of description. However, the spirit, core configuration, and action of the present invention are not limited only to a configuration or action described in the following embodiment. In describing the present invention, a detailed description is omitted when the detailed description of known technologies or configurations related to the present invention is determined to unnecessarily obscure a gist of the present invention

In an embodiment of the present invention, terms including ordinals, such as “first” and “second,” are used only for distinguishing one component from another component, and a term of a singular number includes its plural number unless clearly indicated otherwise in context. In addition, in an embodiment of the present invention, it should be understood that terms such as “configured,” “include,” and “have” do not preclude the presence or addition of one or more other features, numbers, stages, operations, components, parts, or combinations thereof. In addition, in an embodiment of the present invention, a “module” or “unit” may perform at least one function or operation, may be implemented in hardware or software, or may be implemented as a combination of hardware and software, and may be integrated into at least one module and implemented by at least one processor. In addition, in an embodiment of the present invention, at least one of a plurality of elements refers to each one or any combination thereof excluding the remainder among the plurality of elements as well as all of the plurality of elements. In addition, an expression such as “configured to (or set to)” is used interchangeably with an expression such as “suitable for”, “having capacity to”, “designed to”, “adapted to”, “made to”, or “capable of”, depending on a situation. The expression “configured to (or set to)” does not necessarily refer only to hardware that is “specifically designed to.” Instead, in some situations, an expression “a device configured to” may indicate that the device performs a corresponding operation together with other devices or parts. For example, a phrase “a processor configured to perform A, B, and C” may indicate a dedicated processor (e.g., an embedded processor) for performing the corresponding operation or a generic-purpose processor (e.g., a central processing unit (CPU) or an application processor) for performing the corresponding operations by executing at least one software program stored in a memory device.

Hereinafter, a preferred embodiment of the present invention is described in detail with reference to the drawings. This description is provided to enable those skilled in the art to which the present invention pertains to easily practice the present invention, and such a description does not limit the spirit and scope of the present invention.

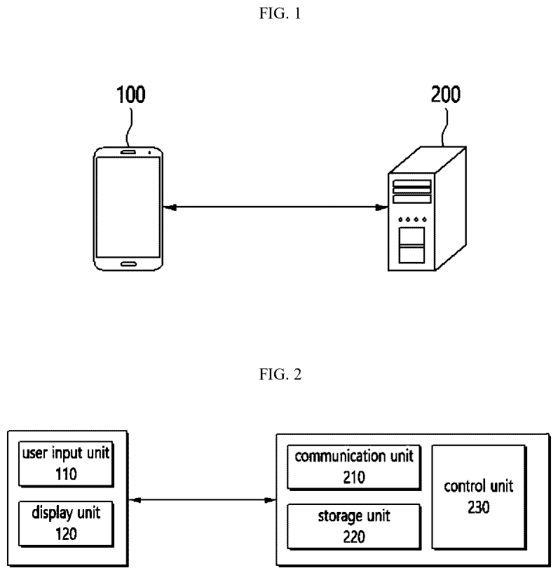

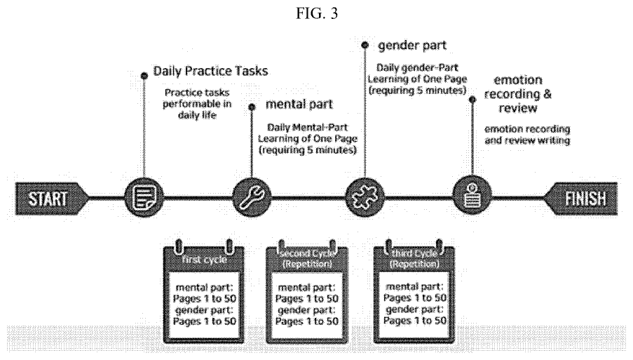

FIG. 1 is a diagram illustrating a schematic configuration of a system for providing mental therapy services to alleviate depression and psychological disorders according to an embodiment of the present invention. FIG. 2 is a diagram illustrating a detailed configuration of the system for providing mental therapy services to alleviate depression and psychological disorders according to an embodiment of the present invention. FIG. 3 is a diagram illustrating a learning flowchart of the system for providing mental therapy services to alleviate depression and psychological disorders according to an embodiment of the present invention.

Referring to FIGS. 1 to 3, a system for providing mental therapy services to alleviate depression and psychological disorders according to an embodiment of the present invention may include a user terminal 100, the user terminal 100 including a user input unit 110 for receiving user input and a display unit 120 for displaying a screen of a pre-installed application in the user terminal 100 to provide the mental therapy services to alleviate depression and psychological disorders. In addition, the system may include a server 200 connected to the user terminal through a network.

Depression and psychological disorders according to an embodiment of the present invention comprehensively indicate states that adversely influence psychological, emotional, and social functions. Depression may include major depressive disorder and may be characterized by symptoms such as chronic sadness, lethargy, loss of interest, low self-esteem, sleep disturbance, reduced concentration, appetite changes, and thoughts of suicide. Depression may be distinguished from a temporary mood drop occurring as a response to an external stimulus and occur through interaction among biological, psychological, and environmental factors.

Causes of depression may include genetic factors, imbalance of neurotransmitters, stress, trauma, chronic diseases, social isolation, economic difficulty, and the like. In addition, depression may manifest differently depending on age, gender, and individual personality traits. For example, in children and adolescents, depression may appear as behavioral changes such as poor academic performance, hyperactivity, and social isolation, and in adults, depression may appear as reduced ability to perform work, interpersonal relationship problems, and the like. In elderly individuals, depressive symptoms related to physical diseases are frequently observed.

Psychological disorders may include not only depression but also anxiety disorders, bipolar disorder, obsessive-compulsive disorder, post-traumatic stress disorder (PTSD), panic disorder, social anxiety disorder, somatic symptom disorder, and dissociative disorder. These psychological disorders may not only cause mental distress, but also interfere with performance of social roles and daily lives of an individual, resulting in significant decline in quality of life. Psychological disorders may not occur due to a single cause and frequently occur as complex results of a biological factor (e.g., genetic predisposition or abnormality in brain structure or function), a psychological factor (e.g., personal experiences, personality, or coping methods), and an environmental factor (e.g., social stress, economic pressure, or family issues).

Depression and psychological disorders are highly likely to become chronic or recur without appropriate therapy and management. In particular, psychological disorders may become chronic if not recognized or treated early, and negatively affect physical health. For example, depression may be highly correlated with physical diseases such as cardiovascular diseases, diabetes, and immune system impairment. In addition, psychological disorders may negatively affect family members and surrounding society and may expand into problems of not only an individual but also an entire social system.

Depression and psychological disorders as defined in the present invention may encompass all states associated with such psychological and emotional distress and are not limited to specific diagnoses or symptoms. Therefore, the present invention may be applied to individuals experiencing various psychological difficulties and has broad applicability regardless of the severity of depression or psychological disorders, a type of manifestation, or individual environmental factors. This feature aims to effectively alleviate various types of psychological difficulties by using a novel mental therapy approach that is used together with or independently from an existing therapy method.

The mental therapy according to an embodiment of the present invention may comprehensively indicate a concept including a method and a process systematically designed to recover and positively improve psychological and emotional states of an individual. The mental therapy may be applied to resolving or alleviating various psychological problems such as depression, anxiety, stress, obsessive-compulsive disorder, and trauma, and in this process, the mental therapy aims to understand principles of emotions, reduce negative emotional habits, and induce positive psychological changes.

The mental therapy may include a self-healing-based approach distinguished from a traditional mental therapy and may be performed based on active participation of a therapy subject. The mental therapy aims to control or overcome emotional changes caused by external factors by analyzing the causes and operating principles of emotions and strengthening an ability to regulate emotions through practice and learning based on such analysis. The mental therapy focuses on enabling the subject to objectively understand emotions of the subject and convert negative emotions into positive emotions.

The mental therapy may include learning data and practice tasks for emotion regulation and may be designed to enable the subject to gradually improve emotional patterns through repetitive processes in which learning and practice are coupled. In detail, the mental therapy may include the following elements.

First, the mental therapy refers to a learning process for understanding basic principles and operating principles of emotions. The learning process may assist in understanding a manner in which emotions are formed and a manner in which emotions are amplified or suppressed by specific events or environments.

Second, the mental therapy refers to a practice process for preventing emotional reactions in specific situations. For example, the subject practices not reacting sensitively to words and actions of others or acquires skills to escape from problematic situations by objectifying emotions of the subject.

Third, the mental therapy refers to a process of forming positive emotional habits through repetitive learning and feedback processes. Repetitive learning provides the same learning data, yet proceeds in a manner of providing transformed sentence structures or expressions to enable a user to newly recognize the content.

The mental therapy may include a stage of recording and analyzing various emotional events occurring in daily life in addition to learning and practice. Through this stage, the subject may identify situations in which the subject experiences negative emotions, understand patterns of repeatedly occurring problems, and learn a method for coping with therewith.

The mental therapy defined in an embodiment of the present invention is not limited to a specific therapy method or technology and includes all processes for finding psychological stability through improvement of the emotional regulation ability. The mental therapy refers to a broad concept applicable regardless of gender, age, cultural background, or severity of psychological state of the subject. The mental therapy according to the present invention may be executed independently or together with an existing drug therapy or psychological counseling, and aims to maximize a self-healing effect based on active participation and continuous learning of a subject. This feature may provide an innovative therapeutic approach for assisting an individual to control emotions and resolve psychological problems on one's own.

An application according to an embodiment of the present invention may comprehensively indicate software installed in the user terminal and providing various functions to alleviate depression and psychological disorders. The application may interact with a user, and may include a system for integrally managing a series of processes including learning-content provision, practice task execution, emotion recording, feedback, and the like.

The application may provide content personalized based on user input data to establish a user-customized therapy environment. The application may receive information such as gender, age, basic emotional state, and psychological characteristics from the user, analyze the information, and propose personalized learning data and practice tasks based on analysis results. For example, learning data including a mental part and a gender part may provide content classified as male-use or female-use based on user gender and provide a learning environment more suitable for the user.

The application may include major functions as follows.

First, the application is provided with a function of providing learning data. The application may provide mental-part and gender-part learning data each for 50 days, and one content may be provided sequentially each day. The user may learn the corresponding content through the application, and understand and improve emotions of the user by responding to questions included in the content.

Second, the application is provided with a function of proposing and managing a practice task. The application may propose the practice tasks capable of being performed in daily life based on content learned by a user, and the user may input and manage whether to perform the practice tasks through the application. The practice tasks may vary depending on user gender, and task difficulty may be adjusted based on a psychological state of the user.

Third, the application is provided with a function of recording emotions and writing reviews. A user may record an emotional state of the user each day and write reviews regarding learning and practice tasks. Written data may be recorded in a storage unit included in the application and subsequently utilized as basic data for data analysis and feedback provision.

Fourth, the application is provided with a function of analyzing data and providing feedback. The application may analyze the stored emotional-record data, practice-task execution data, and learning response data, and may derive emotional-state change trends and learning effects of a user. Analysis results thereof may be provided to the user in the form of graphs and summary text and assist the user to intuitively understand a user state.

The application may be designed to enable anyone to easily access the application through an intuitive and easy-to-use interface. A user may utilize the functions provided by the application without complicated procedures, and each process may be automated to systematically support learning and therapy processes of a user. For example, learning data and practice tasks may be automatically provided, and appropriate feedback may be immediately provided based on the user input data.

In addition, the application may be designed based on repetitive learning. When a first learning cycle is completed, the same learning data may be repeatedly provided for second and third cycles, and questions included in reviews may be provided with transformed vocabulary and sentence structure to enable a user to newly recognize repetitive learning. The application may be designed in this manner to maximize learning effect and to enable a user to continuously participate in a learning process without feeling boredom.

The application according to an embodiment of the present invention is not limited to a specific platform or device, and may operate on various digital devices such as mobile devices, tablets, and desktops. In addition, the application may be linked to a cloud server, stably store and manage data, and support backup and restoration of data. Through this configuration, the application may provide an environment in which a user checks a user state and proceeds with the learning process anytime and anywhere.

The application may be used together with or independently from an existing depression therapy method and aims to maximize the self-healing effect by inducing active participation of a user. This application may propose an innovative software system for assisting in improving emotional states and promoting psychological stability by providing a systematic and repeatable learning environment to a user.

The user terminal 100 according to an embodiment of the present invention may include, for example, a personal computer, a server computer, a handheld or laptop device, a mobile device (e.g., a mobile phone, a personal digital assistant (PDA), or a media player), a multiprocessor system, a consumer electronic device, a mini computer, a mainframe computer, distributed computing including any of the above-mentioned systems or devices, an edge computing environment in which data is processed at an edge where data is generated rather than a central server for data processing, and is not limited only to the above-described examples.

The user terminal 100 may include at least one processor and at least one memory. Here, a processor may include, for example, a central processing unit (CPU), a graphics processing unit (GPU), a microprocessor, an application-specific integrated circuit (ASIC), a field programmable gate array (FPGA), and may have a plurality of cores.

The memory may be a volatile memory (e.g., a random access memory (RAM)) or a non-volatile memory (e.g., a read only memory (ROM) or a flash memory) or a combination thereof. In addition, the user terminal 100 may include an additional storage. The storage may include a magnetic storage or an optical storage, and is not limited thereto. The storage may store a computer-readable instruction for implementing at least one embodiment presented in the specification, and may also store other computer-readable instructions for implementing an operating system, application programs, and the like. The computer-readable instruction stored in the storage may be loaded into the memory to be executed by the processor.

In addition, the user terminal 100 may include the user input unit 110 and an output device. The user input unit 110 may include, for example, a keyboard, a mouse, a pen, a voice input device, a touch input device, an infrared camera, a video input device, another input device, or the like. In addition, an output device may include, for example, at least one display, at least one speaker, at least one printer, another output device, or the like. In addition, a computing device may use an input device or output device disposed in another computing device as the user input unit 110 or the output device. In addition, the computing device may include a communication module that enables the computing device to communicate with another device. Here, the communication module may include a modem, a network interface card (NIC), an integrated network interface, a radio frequency transmitter/receiver, an infrared port, a universal serial bus (USB) connection, or another interface for connecting the computing device to another computing device. The communication module may include wired connection or wireless connection.

Respective components of the user terminal 100 may be interconnected by various interconnections such as a bus (e.g., peripheral component interconnect (PCI), USB, firmware (IEEE 1394), or an optical bus structure) or may be interconnected through a network. Terms such as “component” and “system” used in the present specification generally refer to a computer-related entity which is hardware, a combination of hardware and software, software, or software being executed.

The user terminal 100 according to an embodiment of the present invention may include the display unit 120, and manner of implementing the display unit 120 is not limited, and may be implemented by using various display methods such as a liquid crystal, a plasma, a light-emitting diode, an organic light-emitting diode, a surface-conduction electron-emitter, a carbon nano-tube, a nano-crystal, or the like. In the case of adopting a liquid crystal method, the display unit 120 may include a liquid crystal display panel, a backlight unit for supplying light to the liquid crystal display panel, and a panel driving unit for driving the liquid crystal display panel. Meanwhile, the display unit 120 may be implemented as a quantum dot light-emitting diode (QLED) panel, which is a self-emitting element, without a backlight unit.

The server 200 according to an embodiment of the present invention may include a communication unit 210, a storage unit 220, and a control unit 230.

The communication unit 210 according to an embodiment of the present invention may communicate with the user terminal 100 or another external electronic device by using a wired or wireless communication method. Therefore, the communication unit 210 may be implemented by using various other communication methods in addition to a connection unit including a connector or a terminal for the wired connection. For example, the communication unit 210 may perform at least one of wireless fidelity (Wi-Fi) communication, Bluetooth communication, Zigbee communication, infrared communication, radio control communication, ultra-wide band (UWB) communication, wireless USB communication, and near field communication (NFC). The communication unit 210 may include communication modules such as a Bluetooth low energy (BLE) module, a serial port profile (SPP) module, a Wi-Fi Direct module, an infrared communication module, a Zigbee module, or a near field communication (NFC) module. In addition, the communication unit 210 may be implemented as a device, a software (S/W) module, a circuit, or a chip.

The communication unit 210 according to an embodiment of the present invention may include various communication modules as described above and may further include an internet of things (IoT) communication module having an IoT network for each telecommunications carrier. The IoT communication module may indicate all IoT communication networks enabling services based on various platforms in which a plurality of objects having separate communication units 210 are interconnected through a network. Using the IoT communication module may provide a smoother communication network within a set region.

The storage unit 220 according to an embodiment of the present invention may receive information from the user terminal 100 through the communication unit 210 or receive information from an external search platform and store the same, and may receive text information and image information included in a plurality of webpages received by the control unit 230 and store the information. The storage unit 220 may store various data according to processing and control of the control unit 230 described below. The storage unit 220 may be accessed by the control unit 230 and may perform reading, recording, modifying, deleting, and updating of data. The storage unit 220 may include a non-volatile memory such as a flash memory, a hard disk drive, or a solid-state drive (SSD) to preserve data regardless of whether system power is supplied to the server 200. In addition, the storage unit 220 may include a volatile memory such as a buffer or a RAM for temporarily loading data processed by the control unit 230.

The control unit 230 according to an embodiment of the present invention may perform control for operating respective configurations included in the server 200. The control unit 230 may include a control program (or instruction) for performing the control operations, a non-volatile memory in which the control program is installed, a volatile memory into which at least a part of the installed control program is loaded, and at least one processor or a central processing unit (CPU) for executing the loaded control program. In addition, such a control program may be stored not only in the server 200 but also in another external electronic device.

The control program may include a program(s) implemented as at least one of a basic input/output system (BIOS), a device driver, an operating system, a firmware, a platform, and an application program (i.e., an application). In an embodiment, the application program may be pre-installed or stored in the server 200 during manufacturing of the server 200, or may receive application data from an external source during subsequent use and be installed in the server 200 based on the received data. Application program data may be downloaded, for example, to the server 200 from an external server such as an application market according to a platform of the present invention, and is not limited thereto. Meanwhile, the control unit 230 may be implemented as a device, a software (S/W) module, a circuit, a chip, or a combination thereof.

The control unit according to an embodiment of the present invention may control storing of learning data classified into the mental part and the gender part, and provide mental-part and gender-part learning data classified as male-use and female-use based on gender data input by the user according the same curriculum order.

The control unit according to an embodiment of the present invention may control storing of learning data classified into the mental-part and gender-part learning data classified as male-use or female-use based on the gender data input by the user according to the same curriculum order. The control unit may include a data analysis module for processing the user input data input from the user terminal, a communication module for transmitting learning data to the user terminal, and an execution module for controlling the learning process based on sequential provision of learning data and the user input data.

The control unit may first receive initial information including the gender data from the user. The gender data input by the user may be processed by the data analysis module included in the control unit and used as a reference for selecting learning data suitable for the user. For example, although the mental part and the gender part are included in the same curriculum order, each content may be stored by being classified as male-use or female-use. The mental part may include content for improving emotion regulation and self-recognition ability, and, based on user gender, male-use content may be designed to emphasize objectivity of emotional expression and analytical ability, while female-use content may be designed to emphasize positive conversion of emotions and psychological stability. The gender part include content related to interpersonal relationships, gender awareness, and self-development, and male-use content may focus on healthy management of gender energy and formation of positive social relationships, while female-use content may strengthen self-esteem and relationship recovery skills.

The control unit may execute a curriculum management algorithm to sequentially provide learning data based on the gender data of the user. For example, when the mental-part and gender-part learning data are each stored for 50 days, the control unit may check a learning-progress state and provide the male-use content of the mental part on the first day, and then provide the male-use content of the gender part as a second learning stage on the same day. When the learning process is completed, the control unit may induce recording of practice task execution through the user input unit and control transition to emotion recording and review writing stages. The control unit may be designed to enable learning data, the practice tasks, the emotion recording, and the review writing to proceed in a predetermined order, and provide an environment in which the user conveniently inputs and checks data during the learning process.

In addition, the control unit may manage provision of learning data in the same curriculum order. When the user completes the first learning cycle, the control unit may repeatedly provide the same learning data for the second and third cycles and transform the sentence structure and vocabulary of existing question data for review writing to enable the user to newly immerse in repetitive learning. To this end, the control unit may transform the existing question data by utilizing a grammar-analysis algorithm and a synonym-substitution module, and when transformed question data is identified as semantically similar to existing questions while being expressed differently, the control unit may provide the transformed data as final data.

The control unit according to an embodiment of the present invention may not only provide learning data but also include a function of controlling storage of user data and analyzing the user data to track effects of the learning process and provide feedback. In a process of providing male-use content or female-use content based on the gender data input by the user, the same curriculum order may be maintained to ensure consistent user experience, and gender-specific customized learning data may be provided to maximize learning effects. Such a structure may compensate for limitations in existing mental therapy methods and contribute to maximizing repetitive learning effects in a self-learning environment.

The control unit according to an embodiment of the present invention may provide the user terminal with the mental-part learning data classified as male-use or female-use based on user gender, provide the user terminal with the gender-part learning data classified as male-use or female-use after learning of the mental-part learning data, provide the user with practice tasks performable in daily life, record data input through the user input unit for receiving information on a practice-task execution state, and control a daily learning process to be completed by displaying an input screen on the user terminal for emotion recording and review writing after completion of learning of the gender-part learning data and the mental-part learning data.

The control unit may first receive the initial information including the gender data from the user and store the information in the storage unit, and classify and manage the mental-part and gender-part learning data based thereon. For example, the mental part may include the content focusing on emotion regulation and emotional stability, and male-use content may include content for strengthening logical analysis of emotions and coping skills, whereas female-use content may be designed to induce positive conversion of emotions and psychological stability. The gender part may focus on relationship management and improvement of self-recognition, and male-use content may include content focusing on social role execution and gender energy management, whereas female-use content may include content focusing on self-esteem and relationship recovery.

The control unit may control sequential provision of learning data, and the daily learning process may include the following stages.

First, the control unit may provide the mental-part learning data to the user terminal, and the data may be classified as male-use content or female-use content based on user gender. For example, a male user may be provided with learning data for analyzing causes of emotional occurrence in specific situations and finding coping methods, and a female user may be provided with learning data for alleviating emotional stress and strengthening self-positivity.

Second, when mental-part learning is completed, the control unit may provide the gender-part learning data to the user terminal. The gender-part learning data may also be classified as male-use or female-use based on user gender, and a male user may be provided with content including concrete practice methods for positive conversion of gender energy, and a female user may be provided with learning data focusing on self-esteem and improvement of social relationships.

Third, the control unit may propose practice tasks performable in daily life to the user after completion of a learning stage. For example, in the mental-part learning, the control unit may provide practice tasks such as “responding logically without reacting emotionally to words of others” to enable the user to apply emotional regulation techniques learned in daily life. In the gender-part learning, the control unit may propose specific and executable tasks such as “finding positive intentions in words of others” or “recording and analyzing one's emotions in writing.”

Fourth, the control unit may receive the information on the practice-task execution state of the user through the user input unit and record corresponding data in the storage unit. Input data may be used to analyze the practice-task execution rate of the user and evaluate a learning effect. Practice-task data may be stored for each date and sequentially used as basic data for analyzing emotional-state change trends.

Finally, the control unit may display an emotion recording and a review writing screen on the user terminal as a final stage of a daily learning process. The emotion recording may induce the user to record emotional states experienced during a day in detail, and review writing may provide feedback based on learning data and practice task execution experience. For example, the control unit may provide questions such as “Write the most impressive part among today's learning data” or “What did you feel while performing today's practice task?”

The control unit may be designed to automatically execute all these processes and to enable each stage to be performed in a systematic and repeatable manner. The user may naturally participate in the learning process through the application and gradually improve an emotional state of the user by performing practice tasks and writing emotion recording. These operations of the control unit may include providing customized content based on user gender, managing practice task execution, recording emotional states, and providing feedback, and contribute to maximizing the learning effect.

The control unit according to an embodiment of the present invention may sequentially provide the mental-part learning data and the gender-part learning data by defining the pre-stored mental-part and gender-part learning data each for 50 days as one cycle on a daily basis. The control unit may track a learning-progress state of the user, provide learning data on the daily basis, and control each learning data to be consumed in order. The control unit may structure learning data, store the same in the storage unit, and manage the learning data to be identified for each date and cycle.

In a daily learning process, the control unit may first provide the mental-part learning data, and the corresponding data may include content related to emotion regulation, self-understanding, stress alleviation, and the like. For example, the mental-part learning data may include questions and learning activities such as “Record major emotions you felt today and analyze causes of occurrence of the emotions.” After the user consumes corresponding learning data, the control unit may consecutively provide the gender-part learning data, and the gender part may include content such as relationship management, improvement of self-efficacy, and improvement of social interaction. The gender-part learning data may include activities such as “Record what you felt during a conversation with another person today and think about points to improve.”

When the daily learning process is completed, the control unit may record that corresponding learning data is completed and prepare learning data to be provided on the next day. To this end, the control unit may index learning data stored in the storage unit for each date and update learning progress of the user in real time. When the user does not complete the mental-part learning data or the gender-part learning data, the control unit may be set to provide learning data of a previous stage again. This configuration may ensure consistent curriculum progress even when the user omits or interrupts the learning process.

In addition, the control unit may manage the order of respective learning data to efficiently deliver learning data to the user according to a fixed curriculum. The control unit may control the mental-part learning to be provided prior to the gender-part learning at all times, and control learning data to be provided to the user terminal in a timely manner while maintaining the predetermined order. For example, first data of the mental part may begin with a question that assists emotional recognition, and first data of the gender part may proceed to a simple interpersonal relationship analysis activity. In this manner, the control unit may maintain consistency and efficiency of a learning process.

The control unit may manage learning data in 50-day units and repeatedly provide the same learning data for the second and third cycles when the first learning cycle is completed. To enable the user to approach the same learning data from a new perspective during repetitive learning, the control unit may transform vocabulary and sentence structure of existing learning questions when providing repetitive learning data to the user terminal. For example, when the question “Write your emotions today” is provided in the first cycle, a transformed question such as “Record feelings you experienced throughout the day” may be provided in the second cycle. This configuration may prevent the user from feeling boredom and maximize the learning effect.

In addition, the control unit according to an embodiment of the present invention is designed not only to provide learning data but also to monitor learning progress in real time, prevent data omission or interruption, and optimize learning experience of a user. Through this, a user experiences emotion regulation and self-development in a systematic and repetitive learning environment.

The control unit according to an embodiment of the present invention may provide each learning data by classifying each of the mental-part and gender-part learning data as male-use content or female-use content based on user gender, may repeatedly provide the same learning data for the second and third cycles when the first learning cycle is completed, may classify emotion-record data, learning-response data, and practice-task data input daily by the user for each cycle to store the data in the storage unit, and may identify the learning effect based on comparison among the data stored for each cycle when each cycle is completed.

The control unit according to an embodiment of the present invention may provide male-use content or female-use content of each of the mental and gender parts based on user gender and repeatedly provide the same learning data for the second and third cycles when the first learning cycle is completed. In providing learning data, the control unit may daily collect emotion-record data, learning-response data, and practice-task data input by the user, classify each data for each cycle, and then record the classified data in the storage unit. These data records may be utilized as basic data for tracking the learning progress and analyzing the learning effect.

The control unit may sequentially provide the mental-part learning data and the gender-part learning data when providing the first learning cycle, and select appropriate learning data by classifying male-use content and female-use content based on user gender. For example, male-use content of the mental part may focus on logical analysis of emotions and coping strategies, whereas female-use content may include content inducing self-positive recognition and psychological stability. In the gender part, male-use content may include stress management and social role execution techniques, and female-use content may include techniques for reinforcing relationship recovery and self-esteem.

After the first learning cycle is completed, the control unit may repeatedly provide the same learning data for the second and third cycles. In this process, the control unit may transform question data included in reviews and provide the transformed data to prevent the user from becoming accustomed to or bored with learning data due to repetition. For example, when a question “Write your emotions felt today” is provided in the first cycle, a transformed question such as “Record feelings you experienced during the day” may be provided in the second cycle. This transformation may be performed using the grammar-analysis module and the synonym-substitution algorithm included in the control unit, and final transformed data may be selected by evaluating similarity between respective questions.

The control unit may record data collected in a learning process for each cycle in the storage unit and manage emotion recording, learning responses, and practice task execution states classified for each date and cycle. The stored data may be used as analysis material for evaluating the learning effect. When each cycle is completed, the control unit may compare the stored data and identify the learning effect. For example, by comparing emotion-record data of the first cycle and the second cycle, the control unit may analyze emotional-state change trends of the user and evaluate user participation based on the practice-task execution rate. The learning effect may be quantitatively derived by using indicators such as degree of improvement of emotional states, response-data consistency, and increase in a practice-task execution rate.

The control unit may provide feedback to the user based on such analysis results. For example, when an emotional state change in a certain cycle is insignificant or a practice-task execution rate is low, the control unit may output additional learning guides or motivation messages to the user terminal. Conversely, when the learning effect clearly appears, the control unit may provide positive feedback and induce the user to continuously perform learning.

The control unit according to an embodiment of the present invention may be designed not to remain with simple reuse of content in repetitive provision of learning data but to maximize efficiency of repetitive learning by analyzing the effect based on learning data of the user and providing feedback. Through this configuration, the user may recognize a development state of the user in a learning process and effectively promote psychological stability and self-development.

The control unit according to an embodiment of the present invention may load question data used for review writing in the first learning cycle from the storage unit, extract major phrases (e.g., a subject-verb-object (SVO) structure) included in each question, replace core words among the extracted major phrases with substitutable vocabulary from a synonym database, and rearrange a word order based on substituted words or inserting adverbs, adjectives, or additional phrases to enable the same questions to be recognized differently by the user, thereby generating a plurality of question data to be used for review writing in the second learning cycle in which sentence structures of the question data used for review writing in the first learning cycle are changed.

The control unit according to an embodiment of the present invention may load the question data used for review writing in the first learning cycle from the storage unit and analyze major phrases (e.g., the SVO structure) included in each question to extract core elements such as subject, predicate, and object. The extracted major phrases may be used as basic data for sentence transformation, and the control unit may transform the question data based thereon to deliver the same meanings to the user while being recognized in a new manner

The control unit may first analyze main components of the question data and replace the extracted core words with substitutable vocabulary by utilizing the synonym database. For example, when data “Write your emotions during the day” is used in the first cycle, the control unit may identify core words and phrases such as “emotions,” “write,” and “during the day,” Among these words, “emotions” may be replaced with “feelings” or “sentiments” by using the synonym database, and “write” may be replaced with “record.” Replacement words may be selected by considering priority and contextual suitability in the database.

Based on the replacement words, the control unit may generate transformed question data by rearranging word order or inserting adverbs, adjectives, or additional phrases. For example, a question “Write your emotions during the day” may be provided in a form such as “Record in detail the feelings you experienced during the day” through a transformation process. The transformed sentence may be grammatically correct and designed to enable the user to recognize the corresponding sentence as a newly-expressed sentence while maintaining the same meaning of the existing question.

The control unit may execute a natural-language similarity evaluation algorithm to evaluate the transformed question data. The control unit may calculate semantic similarity between the transformed question data and original question data, and finally select only questions whose similarity is a set threshold (e.g., 80%) or below. Questions with high similarity may be excluded from transformed data, and final transformed question data may be completely prepared to be provided to the user. For example, “Write your emotions during the day” and “Record the feelings you experienced during the day” may be identified as new data because their similarity is the threshold or below.

A plurality of finally generated question data may be provided to the user terminal to enable the user not to recognize the questions as the same as those of a previous cycle during review writing in the second learning cycle and to newly learn the same. The control unit may record the transformed question data in the storage unit, manage the same transformation work to prevent repetition, and support generation of new sentences based on the transformed question data even in subsequent repetitive learning stages.

The operations of the control unit according to this embodiment may maintain the learning effect continuously in a repetitive learning process through transformation of the question data and provide an environment enabling the user to maintain interest in repetitive learning and to immerse oneself. In addition, the transformed question data may be designed to be recognized in a new form while having the same meaning as the existing question and to simultaneously ensure effective utilization of learning data and improvement of user experience. Through this configuration, the control unit may efficiently manage the repetitive provision process of learning data and support a user-customized learning environment.

The control unit according to an embodiment of the present invention may execute a natural-language similarity evaluation between the plurality of question data and the question data used for review writing in the first learning cycle to identify questions whose similarity is equal to or lower than a predetermined threshold as the final transformed question data, and provide the final transformed question data to the user terminal.

The control unit according to an embodiment of the present invention may execute the natural-language similarity evaluation algorithm to compare-analyze the plurality of question data and the question data used for review writing in the first learning cycle. The natural-language similarity evaluation refers to a process for calculating semantic similarity between respective question data, and the control unit may tokenize the question data and measure the similarity by vectorizing each word through a word-embedding technique. Through this configuration, the control unit may select optimal transformed data to prevent the generated question data from becoming excessively similar to the existing question data.

The control unit may utilize machine-learning-based algorithms such as Cosine Similarity, Jaccard Index, or Sentence-BERT to calculate the similarity between the question data. For example, when measuring similarity between existing question data “Write emotions felt today” and transformed question data “Record feelings you experienced during the day,” if semantic similarity between the two sentences is evaluated to be equal to or lower than a threshold (e.g., 80%), the transformed question data may be identified as the final transformed question data.

The control unit may exclude data exceeding the predetermined threshold from the transformed data based on similarity evaluation results. For example, the two sentences “Write emotions during the day” and “Record emotions you felt during the day” have high similarity in terms of a sentence structure and an expression manner, and may be recognized as the same question. Therefore, such data may be excluded from final question data. Meanwhile, “Write in detail the feelings you experienced during the day” has low similarity and may be identified as the final transformed question data.

The final transformed question data may be provided to the user terminal and displayed as a new question when the user writes a review. Before providing the final transformed question data to the user terminal, the control unit may record corresponding data in the storage unit and manage the same to prevent the same data from being repeatedly transformed. This configuration may contribute to preventing redundancy of transformation work in subsequent cycles and improving efficiency of learning data management.

In addition, when providing the final transformed question data to the user terminal, the control unit may record evaluated results as log data to ensure that the transformed questions have the same meanings as the existing questions while being expressed in different forms. This data may be utilized for improvement of learning data and training of additional transformation algorithms. For example, based on user feedback, performance of a question-data transformation algorithm may be adjusted or new synonyms and sentence structures may be added to learning data.

The natural-language similarity evaluation performed by the control unit according to this embodiment may support the user to recognize questions in a new manner in the repetitive provision process of learning data and establish an environment enabling the learning effect to be maximized while maintaining immersion for repetitive learning. Through this configuration, the control unit may efficiently manage the transformation work of learning data and contribute to improvement of user experience.

The control unit according to an embodiment of the present invention may collect emotion-record data, review data, and practice-task execution-state data input through the user input unit, classify the data based on the learning date and cycle, and store the classified data in the storage unit.

The control unit may first collect emotion-record data, transform content written by the user into text data, and assign metadata such as learning date and cycle information. For example, when the user inputs a record “I felt many positive emotions during the day,” the control unit may record the same in the storage unit together with learning date and the cycle information. This data may be sequentially used to analyze emotional-state change trends of the user or evaluate the learning effect.

In the case of review data, the control unit may induce the user to input what the user feels and learns, what needs improvement during the learning process, and the like, and may store corresponding data as text. For example, when the user inputs a review “Emotion regulation method learned today is practically useful,” the control unit may record corresponding data in the storage unit after classifying the review for each date. The review data may user feelings about the learning data, and may thus be usefully utilized for improvement of future learning data or analysis of a learning effect of the user.

The practice-task execution-state data may be managed in a stage of collecting data recording a completion state of the practice tasks performed by the user each day. The control unit may classify practice task execution results input by the user into states such as “completed,” “not completed,” or “partially completed,” and record the input data for each state in the storage unit. For example, when the user inputs “I tried not to react emotionally to others'words today, yet failed in some situations,” the control unit may classify this state as partially completed and record the same. This data may be utilized as important data for analyzing the practice-task execution rate of the user.

The control unit may store the input data based on learning date and cycle to enable each cycle data to be independently analyzed even when the user repetitively learns the same learning data. For example, when the same practice task is recorded as “completed” in the first cycle and recorded as “not completed” in the second cycle, the control unit may compare-analyze this record to identify the practice task execution trends of the user.

The control unit may record the collected data in the storage unit and provide user-customized feedback based on data categorized for each item. For example, when frequency of negative emotions increases in emotion-record data of the user, the control unit may output a customized message such as “Recent emotional state is changing negatively. Apply again emotion regulation methods learned previously” to the user terminal. Conversely, when the practice-task execution rate appears high, the control unit may provide positive feedback such as “Consistent practice task execution is contributing positively to improvement of emotional state.”

The control unit according to this embodiment may systematically store and manage the user input data for each date and cycle, and analyze the learning effect or provide feedback to optimize a learning experience of the user and support psychological improvement based on the stored data. Through this configuration, the user may clearly recognize a learning-progress state of the user and be provided with an environment enabling active participation in the learning process.

The control unit according to an embodiment of the present invention may identify response data corresponding to the same question content for each cycle, calculate the emotional-state change trends and the practice-task execution rates, and output a warning message to the user terminal when at least one of negative emotional states or practice-task non-execution occurs.

The control unit according to an embodiment of the present invention may identify the response data for each cycle corresponding to the same question content and calculate the emotional-state change trends and the practice-task execution rates based thereon. The control unit may compare-analyzes response data for each cycle recorded in the storage unit and derive emotional state change direction of the user and quantitatively calculate the practice-task execution rates. Through this configuration, the control unit may evaluate the learning effect and the user participation and output the warning message to the user terminal when at least one of the negative emotional states or the practice-task non-execution occurs.

To analyze the emotional-state change trends, the control unit may compare the response data input by the user for the same questions in each cycle. For example, when for a question “Write feelings you experienced during the day,” a response “There were many positive feelings” is recorded in the first cycle and a response “There were many negative thoughts” is recorded in the second cycle, the control unit may compare the records and identify that the emotional state changes negatively. Such a change may be derived as an overall emotional state trend by performing time-series analysis based on data for each cycle.

The practice-task execution rate may be calculated based on the practice-task execution-state data input by the user. The control unit may record states such as “completed,” “not completed,” and “partially completed” for the practice tasks provided in each cycle, and calculate the practice-task execution rate by computing a rate to total provided practice tasks. For example, when 6 of 10 practice tasks are recorded as “completed,” a practice-task execution rate may be calculated as 60%.

When emotional state analysis results and the practice-task execution rate fall below a predetermined reference, the control unit may output a warning message to the user. For example, when the emotional state change trend shows a negative direction for three consecutive cycles or the practice-task execution rate appears lower than 50%, the control unit may output a warning message such as “Recent emotional state is changing negatively. Consistently perform practice tasks or review previously learned content.” This message aims to support the user in recognizing the user state and induce more active participation in learning and practice processes.

Simultaneously with outputting the warning message, the control unit may record the corresponding state in the storage unit and sequentially utilize this record for analysis of the learning effect. For example, when the practice-task execution rate increases or the emotional state improves after the user receives the warning message, this record may be utilized as data for evaluating effectiveness of learning data and feedback strategies. Conversely, when a negative state is continuously shown even after the warning message, the control unit may redesign a learning process by providing additional learning data or proposing more specific alternatives to the user.

The control unit according to this embodiment may analyze the emotional state and practice task progress in real time based on the user data, and provide customized feedback capable of maximizing the learning effect. Therefore, the user may clearly recognize learning and practice processes of the user and be provided with a systematic environment that induces continuous psychological improvement.

The control unit according to an embodiment of the present invention may evaluate the learning-progress state of the user based on the data stored after completion of the third learning cycle, calculate the emotional-state change trends and identify the practice-task execution rates and the response-data consistency by comparing emotional-record data of the first, second, and third cycles to quantitatively derive the learning effect of the user, and output the emotional-state change trends and the practice-task execution rates of the user to the user terminal in graph and summary-text forms based on the analysis results of the learning effect.

The control unit according to an embodiment of the present invention may evaluate the learning-progress state of the user based on the stored data after completion of third learning cycle, and quantitatively derive the learning effect of the user by analyzing the emotional-state change trends, the practice-task execution rates, and the response-data consistency. The control unit may classify the emotional-record data collected in the learning process for each cycle and perform time-series comparison based on the corresponding data to calculate emotional-state change patterns. In addition, the practice-task execution rates may be calculated based on the practice-task execution data for each cycle and utilized as indicators for evaluating the learning participation of the user.

To analyze the emotional-record data, the control unit may segment records input by the user for each cycle into a word level and calculate frequency of words representing positive or negative emotions. For example, when an emotional record “Today was calm and happy” is input in the first cycle, the control unit may identify positive-emotion words such as “calm” and “happy” and calculate positive-emotion scores. When a record “Today was full of worries and difficulties” is input in the second cycle, the control unit may identify negative-emotion words such as “worries” and “difficulties” and calculate negative-emotion scores. Based on such data, the control unit may derive the emotional-state change trends for each cycle and quantitatively evaluate whether the emotional states are changed positively or negatively.

The practice-task execution rate may be calculated based on the practice-task execution-state data input by the user. The control unit may calculate a rate of completed practice tasks in each of the first, second, and third cycles, and compare practice-task execution rates of the respective cycles to analyze whether participation in practice tasks is increased or decreased. For example, when a practice-task execution rate of 60% is recorded in the first cycle and increased to 80% in the third cycle, the control unit may identify that participation in practice tasks is improved and determine this increase as a positive learning effect.

The response-data consistency may be evaluated by comparing responses provided by the user for the same questions in each cycle. The control unit may compare-analyze the response data of the user at a sentence-structure level and a major-word level, and check whether the learning effect appears consistently. For example, when responses “It was positive” and “I was happy” are input in the first and third cycles respectively for the same question “Write emotions you felt today,” the control unit may classify these records as positive responses and determine that the learning effect is consistent. In contrast, when responses are substantially contradictory for each cycle, the control unit may identify that the learning effect is inconsistent.

The analysis results of the learning effect may be visualized in the graph and summary-text forms to enable the user to intuitively understand the results. The control unit may represent emotional-state change trends as line graphs based on a time axis and represent practice-task execution rates as bar graphs for each cycle. For example, when the emotional state changes positively, the emotional state change may be displayed as an upward curve in the graph, and when practice-task execution rate increases, a bar-graph height may be gradually increased for each cycle. The summary text may include specific numerical values and analysis results such as “During third learning cycle, frequency of positive emotions increased by 20% and practice-task execution rate improved from 60% to 80%,” and may be output to the user terminal.

Simultaneously with outputting analysis results to the user terminal, the control unit may record the results in the storage unit to enable the user to review data or utilize the results for additional analysis even after completing the learning process. For example, when the user begins the next learning session, customized learning data may be provided or new practice tasks may be proposed based on previous learning-result data.

The control unit according to this embodiment quantitatively may derive the learning effect and intuitively visualize this effect to the user, thus enabling the user to clearly understand the learning-progress state of the user and to more actively participate in the learning and practice processes. Through this configuration, the control unit may maximize effectiveness of learning data and provide an environment capable of supporting continuous psychological improvement.

The control unit according to an embodiment of the present invention may establish correlations among a plurality of variables based on learning data of the user to evaluate a psychological-recovery state of the user, and utilize the evaluation for learning-content provision and feedback generation based thereon.

Meanwhile, a reason why the control unit establishes the correlations among variables is to analyze a learning state and psychological changes of the user in multiple dimensions and precisely evaluate a comprehensive learning effect and a psychological-recovery process, which are difficult to identify through single-variable analysis. Through this configuration, the control unit may maximize learning efficiency and implement technological effects capable of providing a user-customized learning experience.

Learning data of the user may include various elements, and each variable not only has individual meaning but also describes the learning state of the user more accurately through interaction among other variables. For example, when the positive-word rate in emotional records is high while the practice-task execution rate is low, this state may indicate that the user merely records the positive state and does not proceed to an actual behavior. Conversely, when emotional volatility is low and the practice-task execution rate is high, this state may indicate that the user experiences a stable psychological-recovery process through learning. In this context, it is difficult to accurately determine the learning effect through single-variable analysis alone, and it is therefore necessary to establish correlations among a plurality of variables and analyze the correlations in multiple dimensions.

The control unit may collect learning data of the user, normalize the data, and convert the data into an analyzable form.

In emotional records, the positive-word rate and the practice-task execution rate may be set to proportional relationships with the learning effect. When the positive-word rate is high and the practice-task execution rate continuously increases, this rate indicates that the user experiences positive psychological changes through learning.

Learning time may be set to an inversely proportional relationship with the learning effect. When the learning time becomes excessively long, the learning efficiency may decrease due to accumulated fatigue, and learning time exceeding an appropriate range may be evaluated to negatively affect the learning effect.

A learning interval may be set to a logarithmic proportional relationship. When the learning interval is too short or too long, the learning effect may deteriorate, and the learning effect may be evaluated to be maximized when an appropriate interval is maintained.

The number of repetitive learning may be set to have an exponentially proportional relationship with the learning effect. Although an initial number of repetitive learning may sharply increase the learning effect, from a predetermined threshold or above, the learning efficiency may reach a saturation state and an effect of additional repetitive learning may decrease.

The control unit may identify the plurality of variables based on the established correlations and derive a “psychological recovery index (PRI).”

The control unit may analyze the emotional-record data to evaluate the positive-word rate, couple the evaluation with the practice-task execution rate to determine learning immersion of the user, evaluate whether the learning time does not exceed the appropriate range and whether the learning interval is maintained to measure the learning efficiency, check the number of repetitive learning to prevent learning-efficiency degradation caused by excessive repetition, and determine whether an appropriate level of repetitive learning is performed.

The psychological-recovery index according to the present invention refers to an indicator for comprehensively representing the learning effect of the user, and is utilized for the learning-content provision and the feedback generation. For example, when a high psychological-recovery index is derived, the control unit may provide feedback such as “The learning effect is currently very positive. Maintain the same learning pattern.” Conversely, when a low index is derived, the control unit may provide feedback such as “Recent learning interval is excessively short and learning efficiency has decreased. It is recommended to adjust the next learning session to two days later.”

Such a correlation-based analysis may enable comprehensive evaluation of the learning state and psychological-recovery process of the user beyond merely evaluating individual data. Through this configuration, the learning effect may be maximized, individual-customized learning data and feedback may be provided, and multi-dimensional analysis and utilization of the learning data of the user that existing technologies do not solve may be implemented. In particular, the control unit may enhance accuracy and reliability of the learning-management system and support the user in participating more effectively in the learning process in consideration of complex interactions among the data.

As set forth above, the embodiments provide the self-healing-based systematic learning environment in which a patient is able to receive recovery motivation on one's own.

The embodiments provide the system for providing the mental therapy services to alleviate depression and psychological disorders, which may provide learning data classified into a mental part and a gender part in a customized manner based on gender, thereby supporting a patient to understand principles of emotions and improve emotional state through learning data suitable for a patient situation.

The embodiments provide the system for providing the mental therapy services to alleviate depression and psychological disorders, which may include repetitive and systematic processes such as practice tasks, emotion recording, and review writing based on daily learning data, thereby establishing an environment allowing a patient to actively participate in a therapy process, and may repeatedly provide the same curriculum after the completion of the first learning cycle and provide modified review-writing questions to allow a patient to newly recognize learning data, thereby maximizing the learning effect while preventing the patient from feeling boredom.

The embodiments provide the system for providing the mental therapy services to alleviate depression and psychological disorders, which may allow a patient to proceed learning and practice tasks by investing a short time each day to experience improvement of an emotional state and the psychological stability, and furthermore allow the patient to clearly recognize the recovery state of the patient and determine whether further learning or repeated learning is required on one's own by visually providing the learning effect at the completion time point of the third learning cycle, thereby presenting the new self-healing-based depression therapy method for effectively resolving problems in the existing therapy methods.

What is claimed is:

1. A system for providing mental therapy services to alleviate depression and psychological disorders, the system comprising:
a user terminal; and
a server connected to the user terminal through a wired or wireless network,
wherein the user terminal includes:
a user input unit configured to receive user input; and
a display unit configured to display a screen of a pre-installed application in the user terminal to provide the mental therapy services to alleviate depression and psychological disorders,
wherein the server includes:
a storage unit configured to store data of the application;
a communication unit configured to transmit and receive the data to and from the user terminal; and
a control unit configured to control at least one of operations of the user terminal and the server, and
wherein the control unit is configured to:
control storing of learning data classified into a mental part and a gender part, and provide mental-part learning data and gender-part learning data classified as male-use or female-use based on gender data input by a user according to the same curriculum order;
provide the user terminal with the mental-part learning data classified as male-use or female-use based on user gender;
provide the user terminal with the gender-part learning data classified as male-use or female-use based on user gender after learning of the mental-part learning data;
provide the user with practice tasks performable in daily life, and record the data through a user input unit for receiving information on a practice-task execution state; and
control a daily learning process to be completed by displaying an input screen on the user terminal for emotion recording and review writing after completion of learning of the gender-part learning data and the mental-part learning data,
wherein the control unit is configured to:
sequentially provide the mental-part learning data and the gender-part learning data by defining the pre-stored mental-part and gender-part learning data each for 50 days as one cycle on a daily basis;
provide each learning data by classifying each of the mental-part and gender-part learning data as male-use content or female-use content based on user gender;
repeatedly provide the same learning data for second and third learning cycles when a first learning cycle is completed;
classify emotion-record data, learning-response data, and practice-task data input daily by the user for each cycle to store the data in the storage unit; and
identify a learning effect based on comparison among the data stored for each cycle when each cycle is completed, and
wherein the control unit is configured to:
load question data used for review writing in the first learning cycle from the storage unit, extract major structures (e.g., a subject-verb-object (SVO) structure) included in each question, replace core words among the extracted major structures into substitutable vocabulary from a synonym database, and rearrange a word order based on substituted words or inserting adverbs, adjectives, or additional phrases to enable the same question to be recognized differently by the user, thereby generating a plurality of question data to be used for review writing in the second learning cycle in which sentence structures of the question data used for review writing in the first learning cycle are changed; and
execute natural-language similarity evaluation between the plurality of question data and the question data used for review writing in the first learning cycle to identify questions whose similarity is equal to or lower than a predetermined threshold as final transformed question data, and provide the final transformed question data to the user terminal.

2. The system of claim 1, wherein the control unit is configured to:
collect the emotion-record data, review data, and practice-task execution-state data input through the user input unit, classify the data based on the learning date and cycle, and store the classified data in the storage unit; and
identify response data corresponding to the same question content for each cycle, calculate emotional-state change trends and practice-task execution rates, and output a warning message to the user terminal when at least one of negative emotion states or practice-task non-execution occurs.

3. The system of claim 2, wherein the control unit is configured to:
evaluate a learning-progress state of the user based on the data stored after completion of the third learning cycle;
calculate emotional-state change trends and identify the practice-task execution rates and response-data consistency by comparing the emotion-record data of the first, second, and third learning cycles to quantitatively derive the learning effect of the user; and
output the emotional-state change trends and practice-task execution rates of the user to the user terminal by visualizing the trends and the rates in graph and summary-text forms based on analysis results of the learning effect.
